(19) United States
(12) Patent Application Publication
(10) Pub. No.: US 2021/0050113 A1
(43) Pub. Date: Feb. 18, 2021

(54) METHODS AND SYSTEMS FOR RISK ASSESSMENT AND RISK PREDICTION IN OPIOID PRESCRIPTIONS AND PAIN MANAGEMENT TREATMENT

(71) Applicants: Indiana University Research and Technology Corporation, Indianapolis, IN (US); Wake Forest University, Winston-Salem, NC (US); Applied Decision Science LLC, Cincinnati, OH (US)

(72) Inventors: Christopher Albert Harle, Indianapolis, IN (US); Burke Mamlin, Indianapolis, IN (US); Robert W. Hurley, Winston-Salem, NC (US); Nathan Calvert Apathy, Indianapolis, IN (US); Sarah M. Downs, Indianpolis, IN (US); Julie Diiulio, Park Hills, KY (US)

(21) Appl. No.: 16/981,611

(22) PCT Filed: Mar. 15, 2019
(86) PCT No.: PCT/US2019/022571

§ 371 Date: Sep. 16, 2020

Related U.S. Application Data
(60) Provisional application No. 62/643,848, filed on Mar. 16, 2018.

(51) Int. Cl. G16H 50/30; G16H 20/10; G16H 10/60

(52) U.S. Cl. CPC G16H 50/30; G16H 10/60; G16H 20/10

(57) ABSTRACT

A method for identifying and assessing risks for opioid prescriptions is implemented by an opioid analytics server. The method includes receiving a risk assessment profile for analyzing opioid risks for a patient, extracting a first set of health records from a first electronic health records system based on the risk assessment profile, and processing the first set of health records to determine a set of goals, a set of outcomes, and a set of risk factors. The method includes determining, based on the set of goals, the set of outcomes, and the set of risk factors, a plurality of recommended tasks. The method includes providing a user interface including a first region associated with the set of goals and outcomes, a second region associated with the set of risk factors, a third region associated with the plurality of tasks, and a fourth region containing a treatment tracking tool.

### CROSS REFERENCE TO RELATED APPLICATIONS

This application is the national phase application based on PCT/US/2019/022571 (published as WO 2019/178535), filed Mar. 15, 2019, which claims priority to U.S. Provisional Application No. 62/643,848 filed on Mar. 16, 2018, both of which are hereby incorporated by reference in their entireties.

### STATEMENT OF GOVERNMENT SUPPORT

This invention was made with government support under DA046085 awarded by the National Institutes of Health and R01 HS023306 awarded by Agency for Healthcare Research and Quality. The government has certain rights in the invention.

### BACKGROUND OF THE DISCLOSURE

The present disclosure relates generally to methods and systems for predicting risks and assessing risks for opioid prescriptions, and methods and systems for providing information related to opioids and prescriptions of opioids. The present disclosure also relates to providing information related to chronic pain management.

A significant body of evidence has shown that clinical decision support systems can positively impact care delivery, especially process outcomes. Yet, decision support benefits are not automatic and decision support may provide little value or even cause harm. This is unsurprising given that a majority of health information technology projects fail in some way. EHRs and clinical decision support systems are part of complex sociotechnical systems that involve interacting technology, people, and clinical processes. If systems are not designed and implemented with the relevant people and processes in mind, the likelihoods of system non-use, slow-downs, workarounds, and unintended negative effects increase. Recommendations for implementing clinical decision support emphasize system speed, timely information, simplicity, usability, and fit with clinical workflow Similarly, the “5 Rights” of clinical decision support focus on delivering the right information to the right people in the right format through the right channels at the right points in workflow. Unfortunately, these best practices for decision support are inconsistent with the EHRs and information environments in which many clinicians regularly work.

As such, prescribing healthcare providers often face challenges in making judgments and decisions about opioid prescriptions. Opioids are known to present significant risks to at least some patients, and some risks to nearly all patients. For example, when a patient has a history of addiction or mental illness, compromised organ function, or current prescriptions for certain drugs, such a patient may be at serious risk for harm caused by an opioid prescription. In such cases, prescribing healthcare providers ideally seek to prescribe opioids with caution, factoring in health and addiction risks.

Conversely, patients seeking opioid prescriptions often have severe pain issues that may justify a prescription or even an elevated dosage. Given the variety of risk factors and pain profiles associated with patients, physicians face challenges in making a decision about how to approach opioid prescriptions.

These challenges are often exacerbated by limited access to relevant patient data and non-uniform methods of providing relevant patient data. For example, primary care settings have been described as “information chaos” with poorly designed and functioning electronic health record systems and third-party systems that contain pertinent patient data that is not easily accessible. See, e.g., https://www.ncbi.nlm.nih.gov/pubmed/22086819. Further, prescribing healthcare providers often lack training in non-pharmacologic and multidisciplinary approaches to pain care.

Accordingly, systems, methods, and devices for predicting and assessing risks for opioid prescriptions, and methods and systems for providing risk profiles based on these risks are desirable.

### BRIEF DESCRIPTION

In one aspect, a method for identifying risks for opioid prescriptions is provided. The method is implemented by a processor included within an opioid analytics server. The opioid analytics server includes the processor and a memory and is in communication with at least a first electronic health records system. The method includes receiving a risk assessment profile for analyzing opioid risks for a patient, extracting a first set of health records from the first electronic health records system based on the risk assessment profile, and processing the first set of health records to determine a set of goals, a set of outcomes, and a set of risk factors. The method also includes determining, based on the set of goals, the set of outcomes, and the set of risk factors, a plurality of recommended tasks. The method additionally includes providing a user interface including a first region containing information associated with the set of goals and the set of outcomes, a second region containing information associated with the set of risk factors, a third region associated with the plurality of tasks, and a fourth region configured to display a treatment tracking tool.

In a further aspect, a system for identifying risks of opioid prescriptions is provided. The system includes an opioid analytics server having a processor and a memory. The system also includes a first electronic health records system in communication with the opioid analytics server. The processor is configured to receive a risk assessment profile for analyzing opioid risks for a patient, extract a first set of health records from the first electronic health records system based on the risk assessment profile, and process the first set of health records to determine a set of goals, a set of outcomes, and a set of risk factors. The processor is also configured to determine, based on the set of goals, the set of outcomes, and the set of risk factors, a plurality of recommended tasks. The processor is additionally configured to provide a user interface including a first region containing information associated with the set of goals and the set of outcomes, a second region containing information associated with the set of risk factors, a third region associated with the plurality of tasks, and a fourth region configured to display a treatment tracking tool.

In another aspect, an opioid analytics server for identifying risks for opioid prescriptions is provided. The opioid analytics server includes a processor and a memory. The opioid analytics server is in communication with at least a first electronic health records system. The processor is configured to receive a risk assessment profile for analyzing opioid risks for a patient, extract a first set of health records from the first electronic health records system based on the risk assessment profile, and process the first set of health records to determine a set of goals, a set of outcomes, and a set of risk factors. The processor is also configured to determine, based on the set of goals, the set of outcomes, and the set of risk factors, a plurality of recommended tasks. The processor is additionally configured to provide a user interface including a first region containing information associated with the set of goals, the set of outcomes, a second region containing information associated with the set of risk factors, a third region associated with the plurality of tasks, and a fourth region configured to display a treatment tracking tool.

In yet another aspect, an opioid analytics server for identifying risks for opioid prescriptions is provided. The opioid analytics server includes a processor and a memory. The opioid analytics server is in communication with at least a first electronic health records system. The opioid analytics server is configured to extract a second set of health records from a first electronic health record system based on a risk assessment profile, determine from the second set of health records, a first listing of past treatment approaches for chronic pain, and a second listing of current treatment approaches for chronic pain, analyze the first listing and the second listing to determine a third listing of future treatment approaches, analyze the second set of health records to identify risks associated with future treatment approaches, and provide a treatment tracking user interface configured to present at least the first, second, third listing, and a fourth listing of identified risks, and to receive a selection at the third listing of a selected future treatment approach.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 illustrates an exemplary configuration of an opioid analytics server, as described herein.

FIG. 2 illustrates an exemplary user interface provided by the opioid analytics server of FIG. 1.

FIG. 3 illustrates an exemplary report provided by the opioid analytics server of FIG. 1 using the user interface of FIG. 2.

FIG. 4 illustrates an exemplary report provided by the opioid analytics server of FIG. 1 using the user interface of FIG. 2.

FIG. 5 is a flow diagram representing the analytics process from the perspective of the computing device shown in FIG. 1.

FIG. 6 is a diagram of elements of one or more example computing devices that may be used in the system shown in FIG. 1.

FIG. 7 is an exemplary first view of a pain treatment tracking tool provided by the opioid analytics server of FIG. 1 using the user interface of FIG. 2.

FIG. 8A is an exemplary second view of a pain treatment tracking tool provided by the opioid analytics server of FIG. 1 using the user interface of FIG. 2.

FIG. 8B is an exemplary third view of a pain treatment tracking tool provided by the opioid analytics server of FIG. 1 using the user interface of FIG. 2.

FIG. 9A is an exemplary fourth view of a pain treatment tracking tool provided by the opioid analytics server of FIG. 1 using the user interface of FIG. 2.

FIG. 9B is an exemplary fifth view of a pain treatment tracking tool provided by the opioid analytics server of FIG. 1 using the user interface of FIG. 2.

FIG. 9C is an exemplary sixth view of a pain treatment tracking tool provided by the opioid analytics server of FIG. 1 using the user interface of FIG. 2.

FIG. 9D is an exemplary seventh view of a pain treatment tracking tool provided by the opioid analytics server of FIG. 1 using the user interface of FIG. 2.

FIG. 10A illustrates a first view of an exemplary second variation of the user interface provided by the opioid analytics server of FIG. 1.

FIG. 10B illustrates a second view of an exemplary second variation of the user interface provided by the opioid analytics server of FIG. 1.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

The invention seeks to augment the decision-making challenges faced by primary care physicians (PCPs) and other prescribing health care providers by simplifying the pertinent information and making this information actionable and easily accessible. Currently, the electronic health record (EHR) systems that are available often have disorganized or limited data. Further, such EHR systems fail to allow a prescribing health care provider with meaningful information in the context of making an opioid prescription.

The systems and user interfaces described herein were created by a detailed analysis of the requirements of prescribing healthcare providers and, in particular, (a) the information needs in order to support chronic pain treatment, and (b) clinical decision support models required to support chronic pain treatment.

The information needs include at least the following listed below (Table 1):

TABLE 1

| Information Needs | Description |
|---|---|
| Medications | Past and current medications relevant to |
| | pain treatment and related comorbidities. |
| Imaging | Recent imaging (e.g., over the last 6 |
| | months) related to pain organized by body |
| | part. |
| Specialty | Referrals to pain-related specialist; recent |
| utilization | specialist appointments; Indication of |
| | whether referrals and appointments led to |
| | actual encounters. |
| Social determinants | Social determinants of health, such as |
| | insurance status, transportation options, |
| | housing, food access, and patients’ |
| | preferred language. |
| Outcomes and goals | Current pain-related health outcomes (e.g., |
| | pain intensity, physical function, sleep |
| | disturbance); Patient-clinician |
| | goals for pain-related outcomes. |
| Treatment options | Listing of pain treatment options (e.g., |
| | medications, physical therapy, chiropractic, |
| | transcutaneous electrical nerve stimulation, |
| | nutrition, acupuncture, mindfulness); |
| | Context describing rationale for use and |
| | discontinuation for past treatments. |
| Urine drug | For patients prescribed opioids, date and |
| screen results | results of most recent urine drug screen; |
| | Interpretation to identify potential |
| | medication misuse, abuse, or diversion. |
| Prescription drug | For patients prescribed opioids, date and |
| monitoring database | report of controlled substances dispensed to |
| results | patient; Interpretation of results to |
| | identify potential medication misuse, |
| | abuse, or diversion. |

The clinical decision support models required to support chronic pain treatment include at least those listed below (Table 2):

TABLE 2

| Decision Support Models | Description |
|---|---|
| Information accessible | Pain-related information aggregated and |
| in single electronic | organized in a single view in the EHR (e.g., |
| health record location | a patient-level chronic pain dashboard). |
| Information organized | Pain-related information organized in |
| in tables | tables (e.g., a treatment options table or |
| | medication table). |
| Hierarchical information | Pain-related information summarized |
| organization | briefly with interactive capability to drill |
| | down for more details as required (e.g., |
| | clicking on a specialist appointment date to |
| | display a visit note, hovering over a |
| | physical function outcome score displays |
| | an outcome trend over time). |
| Visual cues to | Cues focus clinicians’ attention on relevant |
| focus attention | changes, risks, or needed action (e.g., a |
| | urine drug screen result suggesting |
| | medication misuse, an overdue check of the |
| | prescription drug monitoring report, or a |
| | missed appointment for physical therapy). |
| Predetermined | Provides patient-specific recommendation |
| follow-up | options for follow-up steps based on an |
| | analysis of the range of options that are be |
| | consistent with the patient EHR. |
| Predetermined | Provides patient-specific methods for |
| treatment tracking | tracking treatment that are be consistent |
| | with the patient EHR. |

For example, a prescribing health care provider may be interested in reviewing (i) patient outcome information such as Pain, Enjoyment, General Activity (PEG) scale data, (ii) patient goal information relating to desired outcomes sought by the patient, (iii) contracts associated with an existing opioid prescription, (iv) risk factors associated with a patient, and (v) tasks related to the care of a patient that may be variously not started, incomplete, or completed. Further, a prescribing health care provider may be interested in reports related to this information including, but not limited to, detailed patient medical history and trend reports. Additionally, the prescribing health care provider may be interested in tertiary information such as, for example, prescription drug monitoring program data associated with the patient.

As described herein, the systems, servers, methods, and software described function to provide a prescribing health care provider with a single view, alternatively identified as a “Chronic Pain OneSheet” that may be used for making informed decisions in the prescription of opioids.

The systems, servers, methods, and software described herein may be associated with clinical practice recommendations. In one example, the clinical practice recommendations may be set by the Center for Disease Control and Prevention. In a second example, any suitable clinical care system may be used to define the clinical practice recommendations. The clinical practice recommendations may be used to identify the data that is pertinent for display in the user interface described herein. As such, the clinical practice recommendation may identify the types of information that are relevant to patient goals, patient outcomes, patient risk factors, and patient tasks. The clinical practice recommendation may also indicate the types of tertiary information and reporting that may be provided by the user interface. The clinical practice recommendation may, more generally, be used to define a risk assessment profile. Therefore, in the example embodiment, “risk assessment profile” refers to a knowledge record that informs the clinical practice recommendations associated with prescription of opioids.

As described herein, the systems, servers, methods, and software access health records stored on electronic health record systems and process such information for review by a prescribing health care provider in one centralized user interface. In one example, the systems, servers, methods, and software are co-resident on existing EHR systems and have access to EHR data. In a second example, the systems, servers, methods, and software retrieve EHR data from one or a plurality of external EHR servers. In all embodiments, the user interface functions to provide a singular view for use in opioid prescription. The user interface also provides additional reporting data and allows the prescribing health care provider to complete tasks associated with the prescription.

In the example embodiment, the primary user interface includes at least three regions for information regarding opioid prescription. The first region, associated with patient goals and patient outcomes, includes data associated with (i) patient physical functioning, (ii) patient pain levels according to a suitable pain scale, (iii) patient enjoyment levels, (iv) patient activity levels, and (v) outcomes sought by the patient. The first region may also include access to information associated with a contract related to any existing opioid prescription. Further, the first region is configured to indicate positive ratings and trends. For example, if a patient has low physical functioning, high pain, low enjoyment, or low activity, data presented may include an icon to indicate a warning. In one example, such a warning icon may be presented in the color red. Alternatively, if the patient has medium physical functioning, medium pain, medium enjoyment, or medium activity, data presented may include an icon to indicate such a medium status. In one example, such a medium status may be indicated by the color orange. Similarly, if the patient has high physical functioning, low pain, high enjoyment, or high activity, data presented may include an icon to indicate such a positive status. In one example, such a positive status may be indicated by the color green. The presented icons may further indicate recent trends. For example, if any parameter (i.e., physical functioning, pain, enjoyment, and activity) has increased, an upward arrow icon may be shown with an associated color indicating status. Similarly, if any parameter has decreased, a downward arrow icon may be shown with an associated color indicating status.

In the example embodiment, the second region displays information associated with patient risk factors. The patient risk factor information presented in the user interface may include, for example, a region associated with (i) opioid aberrancy, (ii) medication or drug interaction concerns, (iii) psychiatric risks, (iv) drug screening results, (v) opioid dosing data, and (vi) known side effects historically experienced by the patient.

The opioid aberrancy factor may be used to assess a risk of abuse of opioids by the patient. In one example, a survey-based system such as an opioid risk tool may be used. In a second example, a statistical algorithm may be used to score patient risk of aberrant opioid use. In the second region, the risk factors may present with an associated color and icon to indicate risk levels. For example, a numeric score or a color code may show a low, medium, or high risk. In one example, a green icon is used to indicate low risk, a yellow icon is used to indicate medium risk, and a red icon is used to indicate high risk. As such, opioid aberrancy may be indicated using such icons so that a prescribing health care provider can quickly ascertain a risk level. Further, additional data such as a numeric score or underlying reporting may be provided in association with the opioid aberrancy factor.

The medication interaction information identifies known risks between candidate opioids and prescriptions that the patient is known to be using. As such, the systems, servers, methods, and software retrieve patient prescription, order, dispense or administer information from health records and indicate prescriptions associated with interactions with candidate opioids. For example, if a patient is currently taking benzodiazepines, the system may identify a negative drug interaction with some opioids and accordingly indicate a high risk using an appropriate icon such as a red icon. Similarly, if a patient is taking prescriptions that have benign drug interactions, a green icon may be shown. Further, if a patient is taking prescriptions with mild drug interactions, a yellow icon may be shown.

The psychiatric risk information identifies known concerns with the patient that relate to opioid prescription. Some known psychiatric conditions are associated with higher risks when opioids are prescribed. If the systems, servers, methods, and software identify certain psychiatric diagnoses, an appropriate icon may be shown for psychiatric risk according to the red, green, yellow scale. For example, patients with severe psychiatric concerns may indicate high risk in red, those with mild concerns may indicate risk in yellow, and those with minimal concerns may indicate low risk in green.

In some cases, a patient may have taken drug screening tests that are reflected in the electronic health record. For example, the patient may have taken a urine drug screening to determine whether the patient is taking only their appropriate medications at the appropriate doses. Although such drug screenings may vary, the systems, methods, servers, and software retrieve such information from the electronic health record and determine a risk associated with opioid prescription. For example, if a patient has a urine drug screening and is taking a prescribed opioid according to standard procedures, a urine drug screening (UDS) score of below a first threshold level of morphine milligram equivalents (MME) may indicate a low risk that is displayed in green, or any suitable color. If the patient taking a prescribed opioid according to standard procedures has a UDS score above the first threshold level of MME and below a second threshold level of MME, a medium risk may be shown in yellow, or any suitable color. If the patient taking a prescribed opioid according to standard procedures has a UDS score above the second threshold level of MME, a high risk may be shown in red, or any suitable color. However, these examples are illustrative and may vary depending on patient history, patient profiles, and patient treatment plans.

The opioid dosing information indicates known history of opioid abuse such as overdosing. If a patient has a known history of overdose, this risk may be indicated with a red icon, or any suitable color. Similarly, a suspected opioid overdose history may be indicated with a yellow icon, or any suitable color, and a non-existent history of opioid abuse may be indicated with a green icon, or any suitable color.

The side effects information includes (i) known side effects that the patient has experienced with opioids, or (ii) known side effects that the patient may experience with an opioid based on known patient data. If a patient has a relatively high risk of side effects, this risk may be indicated with a red icon. Similarly, a medium or moderate risk of side effects may be indicated with a yellow icon and a low or non-existent risk of side effects may be indicated with a green icon.

In the example embodiment, the third region displays information associated with tasks relevant to the prescription of opioids. The interactive patient task section presented in the user interface may include, for example, a region associated with (i) a prescription drug monitoring program, (ii) medication interaction actions, (iii), scheduling a drug screening, and (iv) viewing a patient report such as a trend report.

In the example embodiment, a fourth region is configured to present a treatment tracking tool, described below in detail. The treatment tracking tool provides a first listing of past pain treatment approaches, a second listing of current pain treatment approaches, and a third listing of possible future approaches. Each of the first, second, and third listings may include, (a) prescription drug treatment information, (b) referral information, (c) information regarding interventions and/or surgeries, (d) information regarding integrative medical approaches, and (e) information regarding exercise and nutrition.

For each of the listings, prescription drug information may include, for example, (a) method of administering the prescription (e.g., oral, topical, etc.), (b) an identifier associated with a medication, (c) an identifier associated with a treated condition, (d) dosage information, (e) usage information, (f) frequency of usage information, and (g) additional notes.

For each of the listings, referral information may include, for example, (a) source of referral, (b) a condition for which the patient was referred, (c) progress of treatment information, and (d) additional notes.

In other examples, additional regions may be provided that present additional interfaces, data, analyses, and options for the prescribing health care provider.

The prescription drug monitoring program task allows a prescribing health care provider to determine whether recent prescription drug monitoring program (PDMP) data has been retrieved according to a pre-defined schedule. PDMP data may be associated with regulatory authorities such as state authorities. The prescribing health care provider may need to retrieve such PDMP data from an outside source on a regular schedule, such as an annual schedule. PDMP data reflects information tracked by regulatory authorities indicating misuse of prescriptions by a patient including overfilling prescriptions and obtaining prescriptions improperly. Such PDMP data is therefore relevant to a risk of abuse of an opioid prescription and may be factored into the risk factor information reflected in the risk factor region of the user interface. In one example, the opioid analytics server is configured to determine whether the PDMP data is current and, if not, to provide an icon indicating that an update of PDMP data is needed. In the example embodiment, the user interface allows the prescribing health care provider to note whether the PDMP data is current and to access such PDMP data if it is not current using a “download” icon.

The medication interaction action allows a prescribing health care provider to review changes to a patient prescription plan when a negative drug interaction is determined. For example, if a patient is prescribed a benzodiazepine and an opioid, the user interface may indicate a high risk of medication interaction and allow the prescribing health care provider to review alternative prescription options. In the example embodiment, the prescribing health care provider may accordingly select an “edit” icon to adjust patient prescriptions.

The task region also allows the prescribing healthcare provider to schedule a drug screening such as a UDS. In one example, the opioid analytics server is configured to determine whether the drug screening data is current according to a pre-defined schedule. In such cases, the prescribing healthcare provider may request and schedule a drug screening using an associate option.

The task region also allows the prescribing health care provider to select a report such as a medication and pain trends report. In the example embodiment, the medication and pain trends report provides correlated medication, pain, activity, and goal data over time. The medication and pain trends report may accordingly give the prescribing health care provider insight into the patient's experience with prior opioid prescriptions.

The user interface also includes a region associated with general patient data including, for example, patient identifying data, patient vital statistics, and patient allergies and preferences.

The servers, systems, methods, and software described relate to and utilize patient data. Accordingly, such patient data is utilized in compliance with any suitable regulatory regime including, for example, the Health Insurance Portability and Accountability Act of 1996 (HIPAA). In the example embodiment where the servers, systems, methods, and software are co-resident with existing EHR systems, HIPAA compliance is associated with the EHR system compliance. In alternative embodiments, the servers, systems, methods, and software access external EHR systems in a HIPAA compliant manner.

The user interfaces and systems described in this disclosure reflects a practical application that addresses known technical problems in healthcare information processing, electronic healthcare technology tools, and medical diagnoses and prescriptions and other treatments based on such information and tools. Specifically, the unique and new user interfaces and systems reflect a technological improvement that improves information capture and reduces the risk of incorrect or suboptimal diagnoses and treatments. In particular, the user interfaces are configured to provide patient-specific, evidence-based action recommendations that analyze past and ongoing pain management techniques that results in improvements in electronic health record management and resultantly improvements in diagnostic tools. Specifically, the user interfaces and systems provide follow up recommendations and treatment tracking tools that improve pain diagnosis and determinations in prescription of opioids. The treatment tracking tools are also configured to provide alerts to prescribing health care providers that will inform the provider regarding treatment options that may be unadvisable or dangerous for treatment of a specific patient.

The user interfaces and systems described are not currently known in any EHR system, and provide a practical application that improves EHR-based technologies.

A technical effect of the systems and methods described herein is achieved by performing at least one of the following steps: (a) receiving a risk assessment profile for analyzing opioid risks for a patient; (b) extracting a first set of health records from a first electronic health records system based on a risk assessment profile; (c) processing a first set of health records to determine a set of goals, a set of outcomes, and a set of risk factors; (d) determining, based on a set of goals, a set of outcomes, and a set of risk factors, a plurality of recommended tasks; (e) providing a user interface comprising a first region containing information associated with a set of goals and a set of outcomes, a second region containing information associated with a set of risk factors, and a third region associated with a plurality of tasks; (f) identifying a first opioid risk scoring tool, wherein a first opioid risk scoring tool comprises an algorithm used to analyze a level of risk associated with opioid risks for a patient; (g) applying a first opioid risk scoring tool to a first set of risk factors to determine an opioid risk score; (h) providing an opioid risk score in a second region; (i) extracting a second set of health records from a secondary electronic health records system; (j) processing a first set of health records with a second set of health records to determine a set of goals, a set of outcomes, and a set of risk factors; (k) identifying a prescription drug monitoring program associated with a risk assessment profile and a first set of health records; (l) providing access to information from a prescription drug monitoring program from at least one of a first region and a second region; (m) determining a condition status associated with each of a set of goals, and each of a set of outcomes; (n) providing a user interface comprising a first region containing information associated with a set of goals, and a set of outcomes, wherein a first region is provided including a condition status associated with each of a set of goals, and each of a set of outcomes; (o) determining a condition status associated with each of a set of risk factors; (p) providing a user interface comprising a second region containing information associated with a set of risk factors, wherein a second region is provided including a condition status associated with each of a set of risk factors; (q) identifying at least one additional reporting tool; (r) providing access to a at least one additional reporting tool using a user interface; (s) applying a first set of health records to a at least one reporting tool to generate a trend report; (t) providing access to a trend report using a user interface; (u) generating a trend report, wherein a trend report includes a first report region indicating patient prescriptions over a time period, a second report region indicating patient critical events over a time period, and a third report region indicating information associated with a set of goals over a time period; (v) extracting a second set of health records from a first electronic health records system based on a risk assessment profile; (w) determining from the second set of health records, a first listing of past treatment approaches for chronic pain, and a second listing of current treatment approaches for chronic pain; (x) analyzing the first listing and the second listing to determine a third listing of future treatment approaches; (y) analyzing the second set of health records to identify risks associated with future treatment approaches; and (z) providing a treatment tracking user interface configured to present at least the first, second, and third listing and a fourth listing of identified risks.

The systems and methods described collate and simplify clinically relevant information from disparate electronic sources for prescribing health care providers, such as PCPs, who are managing patients with chronic pain. The described user interface presents actionable information to ease the decision-making burden for providers. The user interface collates data on pain and functioning, recent laboratory tests, patient goals, risk factors, protective factors, and important to-do items related to chronic pain. Once collated, the user interface presents these data in a clear, concise single view with the EHR to aid in decision-making.

As used herein, the term processor refers to central processing units, microprocessors, microcontrollers, reduced instruction set circuits (RISC), application specific integrated circuits (ASIC), logic circuits, and any other circuit or processor capable of executing the functions described herein.

Disclosed herein is a method that includes extracting and modifying patient medical data for presentation in an interactive user interface. Such variations of the patient data and retrieved information may be stored in any format on any storage device in or in communication with the computing devices described herein, subject to applicable regulations. The computing devices can convert the information to a format suitable for storage in reserved memory of an opioid analytics server or an associated device. The reserved memory may exist in the form of the pre-defined element of the device's Electrically-Erasable Programmable Read-Only Memory (EEPROM). The reserved memory resides on the computing devices and is intended and reserved to store certain data.

Before describing in detail embodiments that are in accordance with the present disclosure, it should be observed that the embodiments reside primarily in combinations of method steps, system elements, and device components related to analytics in opioid prescription. Accordingly, the device components, system elements, and method steps have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In this document, relative relational terms such as first and second, top and bottom, and the like may be used solely to distinguish one entity or action from another entity or action without necessarily requiring or implying any actual such relationship or order between such entities or actions.

The terms “comprises,” “comprising,” or any other variation thereof, are intended to cover a non-exclusive inclusion, such that a process, method, article, or device that comprises a list of elements does not include only those elements but may include other elements not expressly listed or inherent to such process, method, article, or device. An element proceeded by “comprises . . . a” does not, without more constraints, preclude the existence of additional identical elements in the process, method, article, or device that comprises the element.

It will be appreciated that embodiments of the disclosure described herein may be comprised of one or more conventional processors and unique stored program instructions that control the one or more processors to implement, in conjunction with certain non-processor circuits, some, most, or all of the functions of preparing a communications device for the opioid analytics methods described herein. The non-processor circuits may include, but are not limited to, a radio receiver, a radio transmitter, signal drivers, clock circuits, power source circuits, and user input devices. As such, these functions may be interpreted as steps of a method to perform preparing a computing device for analytics associated with opioid prescription. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used. Thus, methods and means for these functions have been described herein.

Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation.

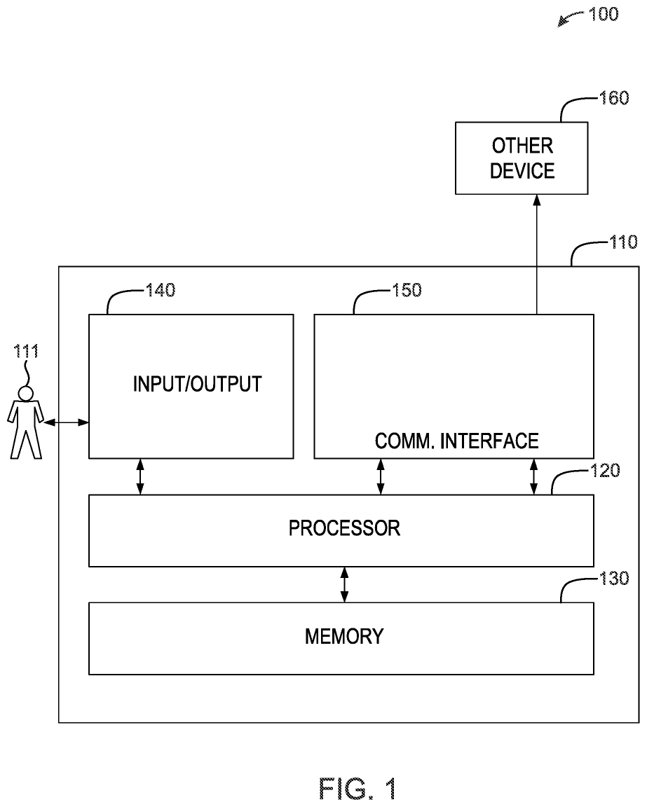

FIG. 1 illustrates an exemplary configuration 100 of a computing device such as the opioid analytics server. Specifically, FIG. 1 illustrates an exemplary configuration 100 of a computing device 110 operated by a user 111 in accordance with one embodiment of the present invention. Computing device 110 may include, but is not limited to a medical computing device, a medical server device, and an electronic health record server. Alternatively, computing device 110 may be any computing device capable of the opioid analytics methods described herein and is generally referred to as opioid analytics server 110 herein. In some variations, the characteristics of the described components may be more or less advanced, primitive, or non-functional.

In the exemplary embodiment, opioid analytics server 110 includes a processor 120 for executing instructions. In some embodiments, executable instructions are stored in a memory area 130. Processor 120 may include one or more processing units, for example, a multi-core configuration. Memory area 130 is any device allowing information such as executable instructions and/or written works to be stored and retrieved. Memory area 130 may include one or more computer readable media.

Opioid analytics server 110 also includes at least one input/output component 140 for receiving information from and providing information to user 111. In some examples, input/output component 140 may be of limited functionality or non-functional as in the case of some wearable computing devices. In other examples, input/output component 140 is any component capable of conveying information to or receiving information from user 111. In some embodiments, input/output component 140 includes an output adapter such as a video adapter and/or an audio adapter. Input/output component 140 may alternatively include an output device such as a display device, a liquid crystal display (LCD), organic light emitting diode (OLED) display, or “electronic ink” display, or an audio output device, a speaker or headphones. Input/output component 140 may also include any devices, modules, or structures for receiving input from user 111. Input/output component 140 may therefore include, for example, a keyboard, a pointing device, a mouse, a stylus, a touch sensitive panel, a touch pad, a touch screen, a gyroscope, an accelerometer, a position detector, or an audio input device. A single component such as a touch screen may function as both an output and input device of input/output component 140. Input/output component 140 may further include multiple sub-components for carrying out input and output functions.

Opioid analytics server 110 may also include a communications interface 150, which may be communicatively coupleable to a remote device such as a first electronic health record system, a remote server, a secondary electronic health record system, or any other suitable system. Communication interface 150 may include, for example, a wired or wireless network adapter or a wireless data transceiver for use with a mobile phone network, Global System for Mobile communications (GSM), 3G, 4G, or other mobile data network or Worldwide Interoperability for Microwave Access (WIMAX). Communications interface 150 is configured to allow opioid analytics server 110 to interface with any other computing device using an appropriate wireless or wired communications protocol such as, without limitation, BLUETOOTH®, Ethernet, or IEE 802.11. Communications interface 150 allows opioid analytics server 110 to communicate with any other computing devices 160. In the example embodiment, other computing device 160 may be the first electronic health record system. In a second embodiment, other computing device 160 is the secondary electronic health record system or a tertiary electronic health record system. In some embodiments, opioid analytics server 110 includes the opioid analytics server and the first electronic health record system. In some further embodiments, other computing device 160 provides access to external data such as PDMP data or UDS data. In all examples, opioid analytics server 110 is configured to appropriately adhere to pertinent regulatory protocol such as HIPAA.

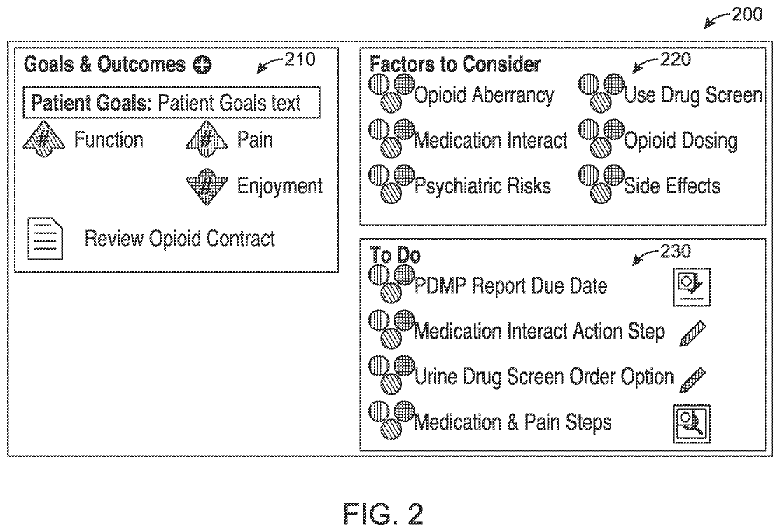

FIG. 2 illustrates an exemplary user interface 200 provided by the opioid analytics server 110 of FIG. 1. As indicated above, user interface 200 includes four regions. A first region 210 indicates patient goal and outcome information. First region 210 is associated with patient goals and patient outcomes, includes data associated with (i) patient physical functioning, (ii) patient pain levels according to a suitable pain scale, (iii) patient enjoyment levels, (iv) patient activity levels, and (v) outcomes sought by the patient. The first region 210 may also include access to information associated with a contract related to any existing opioid prescription. Further, the first region is configured to indicate positive ratings and trends. For example, if a patient has low physical functioning, high pain, low enjoyment, or low activity, data presented may include an icon to indicate a warning. In one example, such a warning icon may be presented in the color red. Alternatively, if the patient has medium physical functioning, medium pain, medium enjoyment, or medium activity, data presented may include an icon to indicate such a medium status. In one example, such a medium status may be indicated by the color orange. Similarly, if the patient has high physical functioning, low pain, high enjoyment, or high activity, data presented may include an icon to indicate such a positive status. In one example, such a positive status may be indicated by the color green. The presented icons may further indicate recent trends. For example, if any parameter (i.e., physical functioning, pain, enjoyment, and activity) has increased, an upward arrow icon may be shown with an associated color indicating status. Similarly, if any parameter has decreased, a downward arrow icon may be shown with an associated color indicating status.

User interface 200 also includes second region 220. Second region 220 displays information associated with patient risk factors. The patient risk factor information presented in the user interface may include, for example, a region associated with (i) opioid aberrancy, (ii) medication or drug interaction concerns, (iii) psychiatric risks, (iv) drug screening results, (v) opioid dosing data, and (vi) known side effects historically experienced by the patient. As described above, second region 220 displays risk factors using suitable icons to indicate according risk levels.

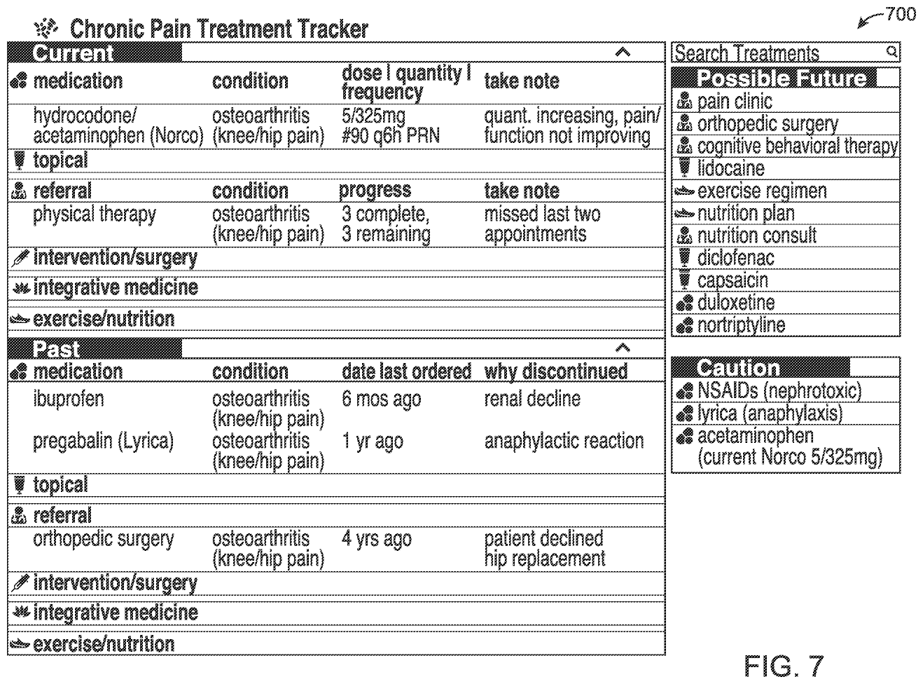

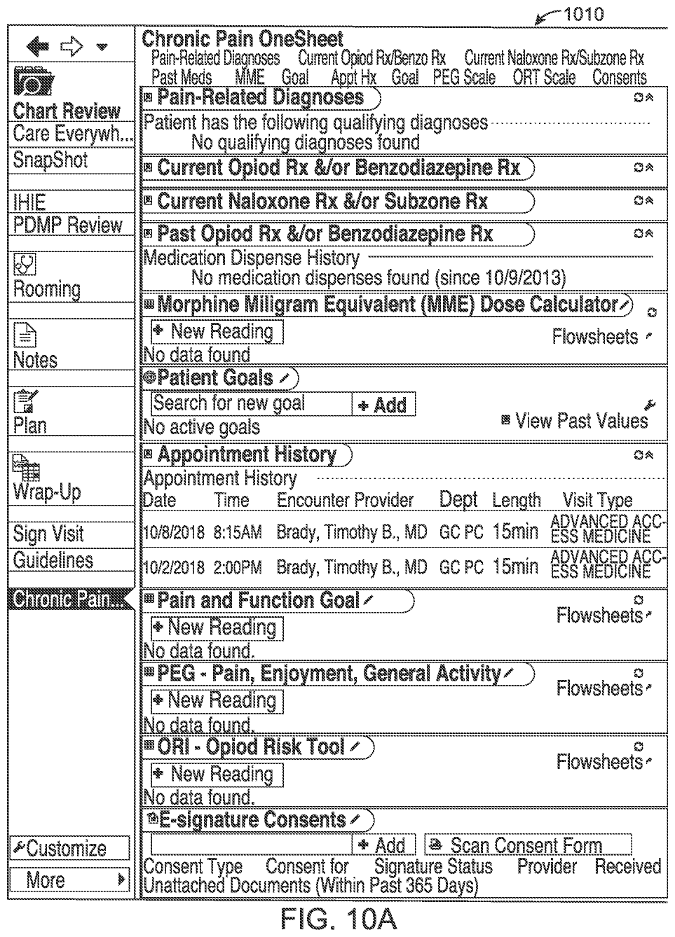

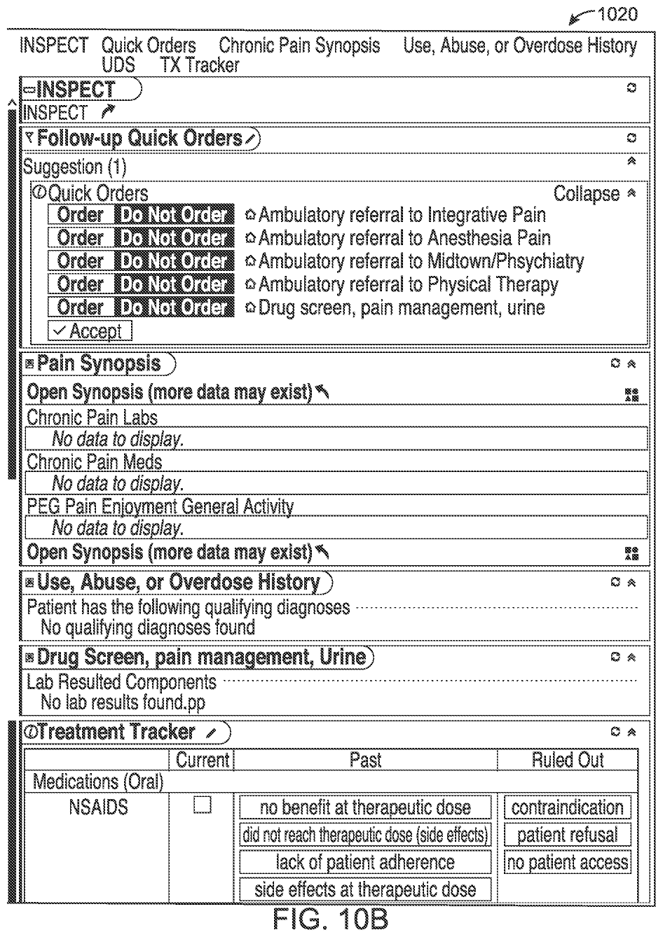

Referring to user interface 200, the third region 230 displays information associated with tasks relevant to the prescription of opioids. The interactive patient task section 230 presented in the user interface 200 may include, for example, options associated with (i) a prescription drug monitoring program, (ii) medication interaction actions, (iii), scheduling a drug screening, and (iv) viewing a patient report such as a trend report. In some examples, user interface 200 is also configured to display treatment tracking tool 700 (see FIG. 7) in a fourth region (shown in FIG. 10B).

The prescription drug monitoring program task allows a prescribing health care provider to determine whether recent prescription drug monitoring program (PDMP) data has been retrieved according to a pre-defined schedule. PDMP data may be associated with regulatory authorities such as state authorities. The prescribing health care provider may need to retrieve such PDMP data from an outside source on a regular schedule, such as an annual schedule. PDMP data reflects information tracked by regulatory authorities indicating misuse of prescriptions by a patient including overfilling prescriptions and obtaining prescriptions improperly. Such PDMP data is therefore relevant to a risk of abuse of an opioid prescription and may be factored into the risk factor information reflected in the risk factor region of the user interface. In one example, the opioid analytics server is configured to determine whether the PDMP data is current and, if not, to provide an icon indicating that an update of PDMP data is needed. In the example embodiment, the user interface allows the prescribing health care provider to note whether the PDMP data is current and to access such PDMP data if it is not current using a “download” icon.

The medication interaction action allows a prescribing health care provider to review changes to a patient prescription plan when a negative drug interaction is determined. For example, if a patient is prescribed a benzodiazepine and an opioid, the user interface may indicate a high risk of medication interaction and allow the prescribing health care provider to review alternative prescription options. In the example embodiment, the prescribing health care provider may accordingly select an “edit” icon to adjust patient prescriptions.

The task region also allows the prescribing healthcare provider to schedule a drug screening such as a UDS. In one example, the opioid analytics server is configured to determine whether the drug screening data is current according to a pre-defined schedule. In such cases, the prescribing healthcare provider may request and schedule a drug screening using an associate option.

The task region also allows the prescribing health care provider to select a report such as a medication and pain trends report. In the example embodiment, the medication and pain trends report provides correlated medication, pain, activity, and goal data over time. The medication and pain trends report may accordingly give the prescribing health care provider insight into the patient's experience with prior opioid prescriptions.

User interface 200 also may include a region (not shown) associated with general patient data including, for example, patient identifying data, patient vital statistics, and patient allergies and preferences.

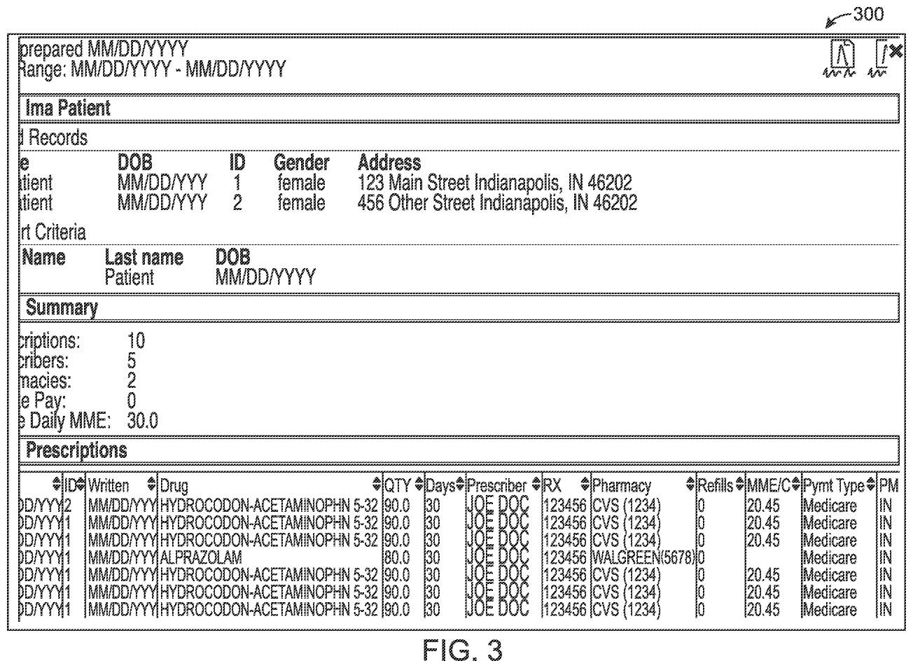

FIG. 3 illustrates an exemplary report 300 provided by the opioid analytics server 110 of FIG. 1 using the user interface 200 of FIG. 2. Specifically, by selecting the red download option associated with “PDMP Report Due Date” in region 230 of FIG. 2, the opioid analytics server 110 may provide access to the PDMP report information and display it according to report 300. As indicated in report 300, the opioid analytics server 110 provides access to data from a prescription drug monitoring program such as a state monitoring program. In the example embodiment, report 300 is provided via a tertiary system hosted by an appropriate state agency. However, report 300 may not be hosted on opioid analytics server 110 unless permitted by the state agency.

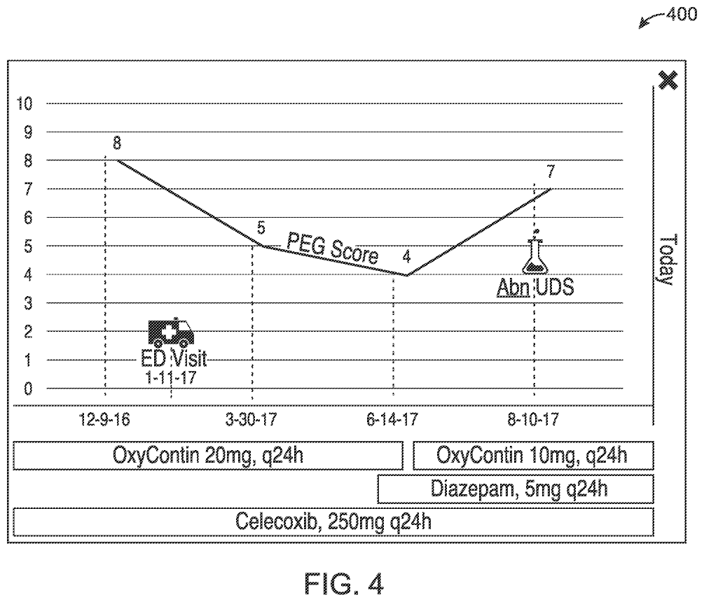

FIG. 4 illustrates an exemplary report 400 provided by the opioid analytics server 110 of FIG. 1 using the user interface 200 of FIG. 2. Specifically, by selecting the green magnifying glass icon associated with “Medication & Patient Trends” in region 230 of FIG. 2, the opioid analytics server 110 accesses a set of health records from the EHR and provides the data by correlating patient trends over time. Specifically, report 400 is a trend graph correlating medication prescriptions, PEG data, and patient critical events over time.

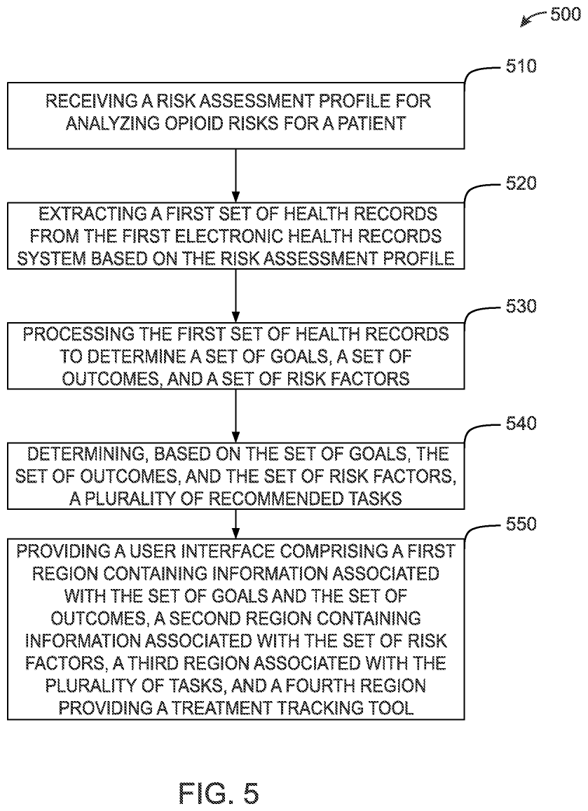

FIG. 5 is a flow diagram 500 representing the analytics process from the perspective of opioid analytics server 110 shown in FIG. 1. Opioid analytics server 110 is configured to perform a method, using processor 120, that includes receiving 510 a risk assessment profile for analyzing opioid risks for a patient and extracting 520 a first set of health records from the first electronic health records system based on the risk assessment profile. The method also includes processing 530 the first set of health records to determine a set of goals, a set of outcomes, and a set of risk factors. The method additionally includes determining 540, based on the set of goals, the set of outcomes, and the set of risk factors, a plurality of recommended tasks. The method further includes providing 550 a user interface comprising a first region containing information associated with the set of goals and the set of outcomes, a second region containing information associated with the set of risk factors, a third region associated with the plurality of tasks, and a fourth region providing a treatment tracking tool. To provide the treatment tracking tool, the opioid analytics server 110 is configured to extract a second set of health records from a first electronic health records system based on a risk assessment profile, determine from the second set of health records, a first listing of past treatment approaches for chronic pain, and a second listing of current treatment approaches for chronic pain, analyze the first listing and the second listing to determine a third listing of future treatment approaches, and provide the treatment tracking tool within the fourth region, wherein the treatment tracking tool is configured to present at least the first, second, and third listing and a fourth listing of identified risks.

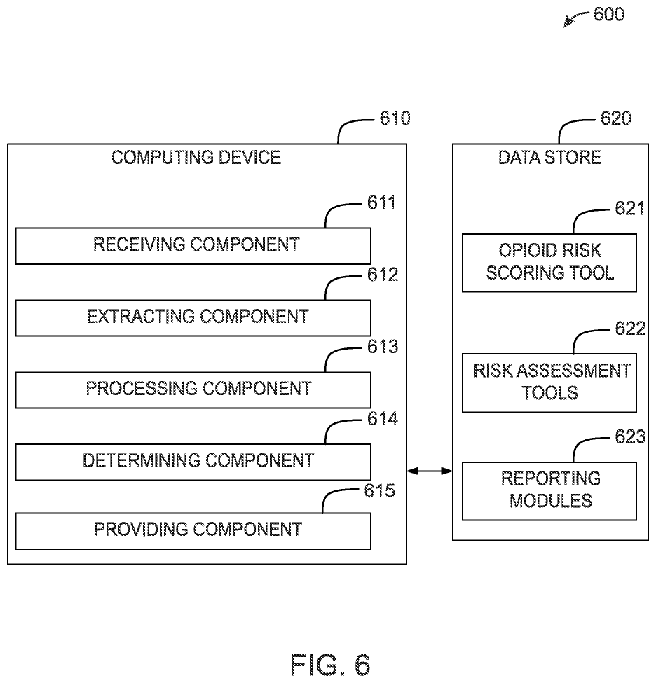

FIG. 6 is a diagram 600 of elements of one or more example computing devices that may be used in the opioid analytics server 110 shown in FIG. 1. In some embodiments, computing device 610 is similar to opioid analytics server 110 shown in FIG. 1.

Data store 620 may be stored at a memory such as memory 130 (shown in FIG. 1) or any other suitable location. Data store 620 may be coupled with several separate components 611, 612, 613, 614, and 615 within computing device 610, which perform specific tasks.

In this embodiment, data store 620 includes opioid risk scoring tool 621, risk assessment tools 622, and reporting modules 623. Computing device 610 may include data store 620, as well as data storage devices (not shown).

Computing device 610 also includes a receiving component 611 for receiving a risk assessment profile for analyzing opioid risks for a patient, an extracting component 612 for extracting a first set of health records from the first electronic health records system based on the risk assessment profile, a processing component 613 for processing the first set of health records to determine a set of goals, a set of outcomes, and a set of risk factors, a determining component 614 for determining, based on the set of goals, the set of outcomes, and the set of risk factors, a plurality of recommended tasks, and a providing component 615 for providing a user interface comprising a first region containing information associated with the set of goals and the set of outcomes, a second region containing information associated with the set of risk factors, and a third region associated with the plurality of tasks.

FIG. 7 is an exemplary first view of a pain treatment tracking tool provided by the opioid analytics server of FIG. 1 using the user interface of FIG. 2. Specifically, a treatment tracking tool 700 (“Chronic Pain Treatment Tracker”) is provided and generated by opioid analytics server 110 as follows. Opioid analytics server 110 extracts a second set of health records from the first electronic health records system based on the risk assessment profile. The opioid analytics server 110 also determines from the second set of health records a first listing of past treatment approaches for chronic pain and a second listing of current treatment approaches for chronic pain. The opioid analytics server 110 also analyzes the first listing and the second listing to determine a third listing of future treatment approaches and analyzes the second set of health records to identify risks associated with future treatment approaches. The opioid analytics server 110 provides a treatment tracking user interface configured to present at least the first, second, third listing, and a fourth listing of identified risks, and to receive a selection at the third listing of a selected future treatment approach.

Treatment tracking tool includes a “current” treatment region, a “past” treatment region, a “future” treatment region, and a “caution” region. The “current” region includes a listing of current pain treatment approaches. In one respect, the “current”, “past”, and “future” regions improve upon existing models by including both medication and non-medication based treatments. As such, the “past” and “current” treatment regions include listings of treatments including (a) medications (including topical, oral, or other forms of administration), (b) referral sources (e.g., physical therapy, pain specialists, and orthopedics), (c) interventions (e.g., surgical interventions, injections, transcutaneous electrical nerve stimulation (“TENS”) units, heat therapy, and ice therapy), (d) integrative medical approaches (e.g., meditation), and (e) lifestyle based treatments (e.g., exercise and nutrition).

In the “current” and “past” treatment regions, treatments in the listings may be grouped according to any suitable method. By default, treatments are grouped by categories identified above—i.e., medications, referral sources, interventions, integrative medical approaches, and lifestyle based approaches. Each of the listings in the “past” and “current” regions of the treatment tracking tool 700 allows for a prescribing healthcare provider to include notes regarding treatment.

The treatment tracking tool 700 provides the “past” region to ensure that the prescribing healthcare provider has access to past pain treatment approaches. Each treatment listed in the “past” region also includes at least (a) a date last ordered, and (b) a reason that the treatment was discontinued. By providing this information, the prescribing healthcare provider may make an informed determination about possible future treatments.

The treatment tracking tool 700 provides the “future” region to provide recommended possible future treatment listings. The “future” treatment region is generated based on opioid analytics server 110 analyzing the first listing and the second listing to determine a third listing of future treatment approaches. Specifically, opioid analytics server 110 is configured to retrieve a list of possible treatments stored within an external health record system. Opioid analytics server 110 compares the list of possible treatments and deprioritizes any possible treatments that have previously been tried and any possible treatments that are inconsistent with the patient profile because, for example, the patient presents risk factors that make such treatment inadvisable.

The treatment tracking tool 700 provides the “caution” region as follows. Opioid analytics server 110 retrieves information from the first and second set of health records. Opioid analytics server 110 retrieves a data repository linking a set of alerts to conditions, allergies, lab result statuses, and diagnoses relevant to pain diagnoses and pain medication. Opioid analytics server 110 compares information retrieved from the first and second set of health records to the data repository and identifies a listing of alerts to be presented in the “caution” region.

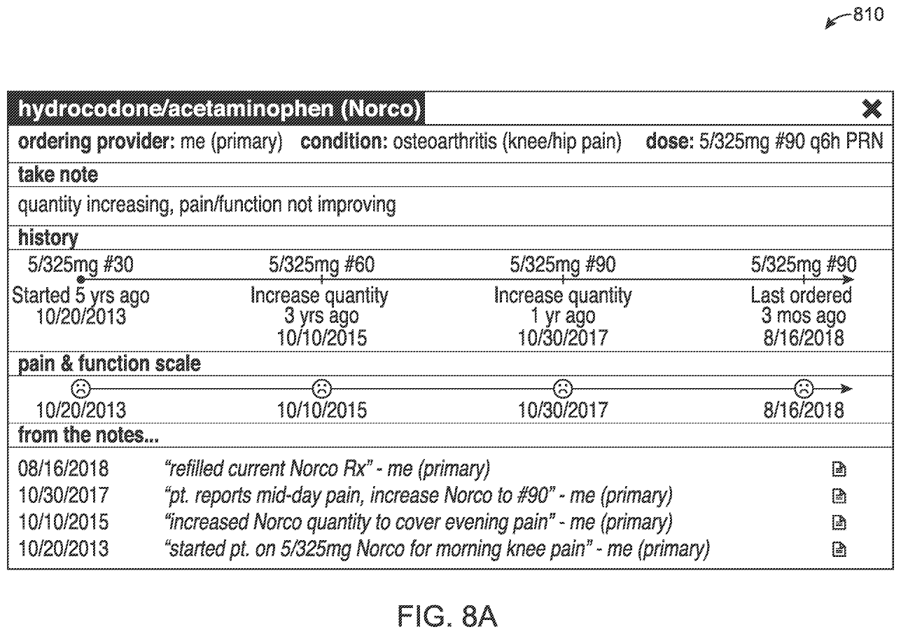

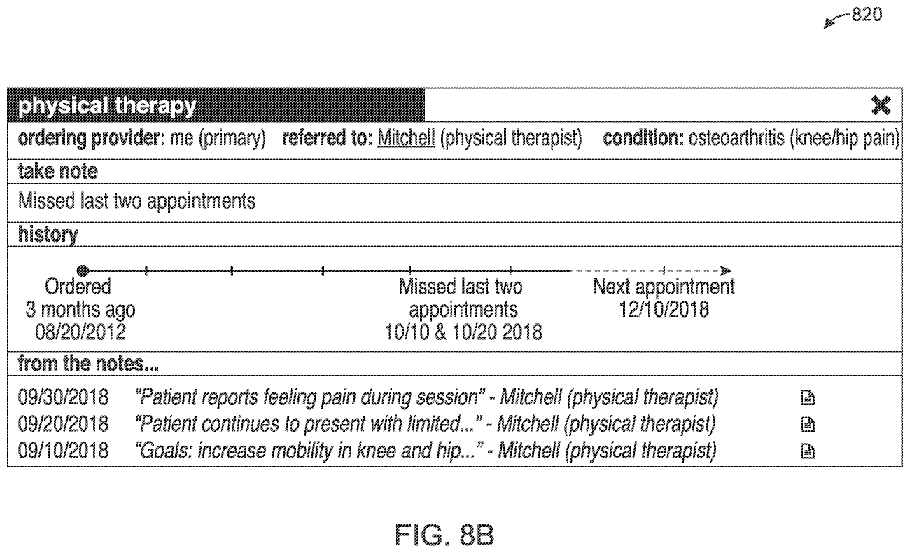

FIG. 8A is an exemplary second view 810 of pain treatment tracking tool 700 provided by the opioid analytics server of FIG. 1 using the user interface of FIG. 2. View 810 provides a graphical display of a patient history of use of a particular prescription. View 810 presents information extracted from first and second set of health records and provides notes, patient usage history, and patient PEG data on a timeline to provide a full history of usage by a patient.

FIG. 8B is an exemplary third view 820 of pain treatment tracking tool 700 provided by the opioid analytics server of FIG. 1 using the user interface of FIG. 2. View 820 provides a full history of patient compliance with related treatments including physical therapy (as shown), integrative medicine, exercise, nutrition, or any other suitable treatment approach.

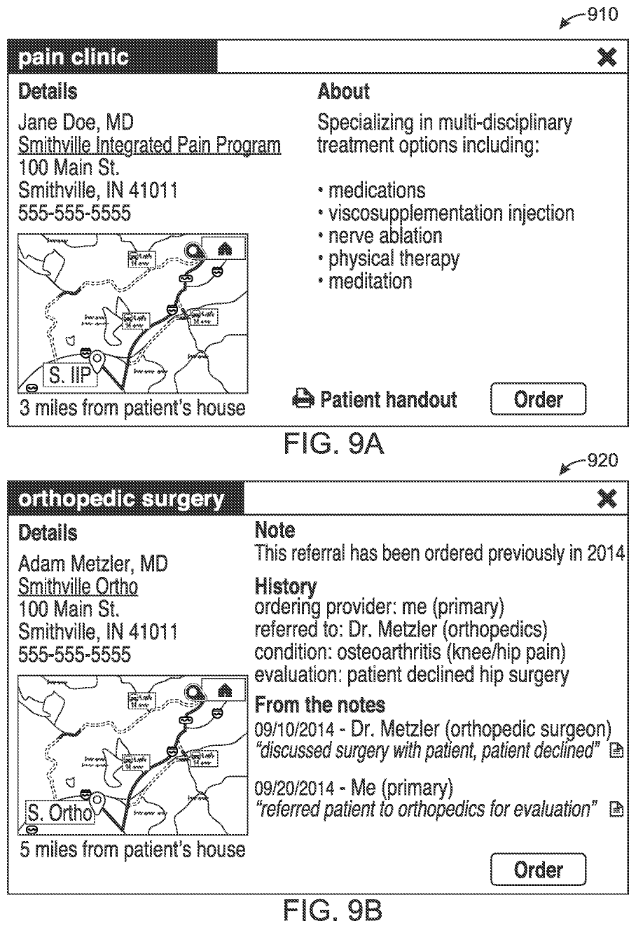

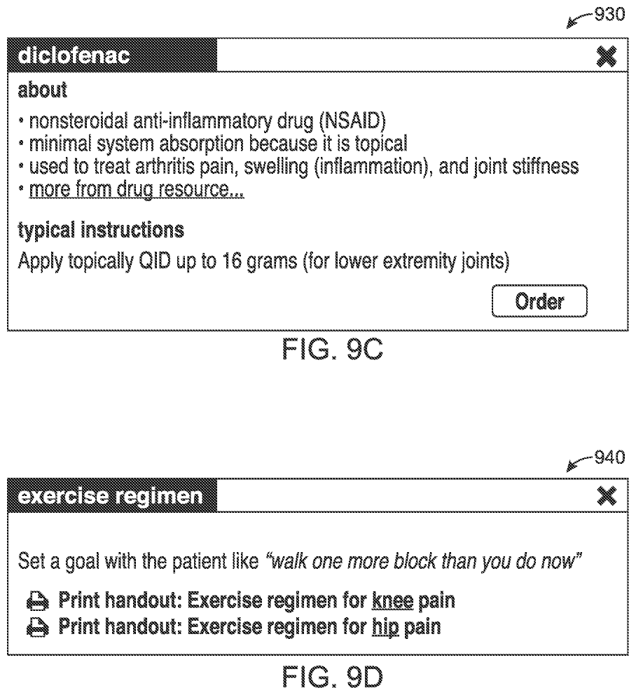

FIGS. 9A and 9B are exemplary fourth and fifth views 910 and 920 of pain treatment tracking tool 700 provided by the opioid analytics server of FIG. 1 using the user interface of FIG. 2. Views 910 and 920 provide information regarding the ease of access by a patient to a particular treatment facility including, for example, a map of distances from a patient's home to a pain clinic or a surgical center. Views 910 and 920 may also present notes from the patient or healthcare providers.

FIG. 9C is an exemplary sixth view 930 of pain treatment tracking tool 700 provided by the opioid analytics server of FIG. 1 using the user interface of FIG. 2. View 930 presents information from drug resources and links to obtain additional information regarding a particular drug.

FIG. 9D is an exemplary seventh view 940 of pain treatment tracking tool 700 provided by the opioid analytics server of FIG. 1 using the user interface of FIG. 2. Seventh view 940 presents supplemental information regarding patient experiences, instructions, and goals.

FIGS. 10A and 10B illustrates a first and second view of an exemplary second variation of the user interface provided by opioid analytics server 110 of FIG. 1. In the examples shown in first view 1010 and second view 1020, the second variation includes a first region (equivalent to region 210) that displays patient goal and outcome information, a second region (equivalent to region 220) displays information associated with patient risk factors, and a third region (equivalent to region 230) that displays information associated with tasks relevant to the prescription of opioids. Additionally, the second variation of the user interface includes a fourth region shown in view 1020 that contains pain treatment tracking tool 700. The second variation of the user interface also includes a pain synopsis area configured to provide a summary of information related to the patient pain treatment and a follow-up area that provides follow-up actions to be provided based on patient data. The follow-up area is configured to allow a prescribing healthcare provider to quickly and efficiently make evidence based follow-up recommendations and referrals as shown in view 1020. The second variation of the user interface also includes a consent region that displays and receives inputs related to consent statements, and an appointment region that documents histories of patient appointments with healthcare providers relevant to pain management. The systems and processes are not limited to the specific embodiments described herein. In addition, components of each system and each process can be practiced independent and separate from other components and processes described herein. Each component and process also can be used in combination with other assembly packages and processes.

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

While the disclosure has been described in terms of various specific embodiments, those skilled in the art will recognize that the disclosure can be practiced with modification within the spirit and scope of the claims.

As will be appreciated based on the foregoing specification, the above-described embodiments of the disclosure may be implemented using computer programming or engineering techniques including computer software, firmware, hardware or any combination or subset thereof. Any such resulting program, having computer-readable code means, may be embodied or provided within one or more computer-readable media, thereby making a computer program product, i.e., an article of manufacture, according to the discussed embodiments of the disclosure. Example computer-readable media may be, but are not limited to, a flash memory drive, digital versatile disc (DVD), compact disc (CD), fixed (hard) drive, diskette, optical disk, magnetic tape, semiconductor memory such as read-only memory (ROM), and/or any transmitting/receiving medium such as the Internet or other communication network or link. By way of example and not limitation, computer-readable media comprise computer-readable storage media and communication media. Computer-readable storage media are tangible and non-transitory and store information such as computer-readable instructions, data structures, program modules, and other data. Communication media, in contrast, typically embody computer-readable instructions, data structures, program modules, or other data in a transitory modulated signal such as a carrier wave or other transport mechanism and include any information delivery media. Combinations of any of the above are also included in the scope of computer-readable media. The article of manufacture containing the computer code may be made and/or used by executing the code directly from one medium, by copying the code from one medium to another medium, or by transmitting the code over a network.

All of the patents, patent applications, patent application publications and other publications recited herein are hereby incorporated by reference as if set forth in their entirety.

The present inventive concept has been described in connection with what are presently considered to be the most practical and preferred embodiments. However, the inventive concept has been presented by way of illustration and is not intended to be limited to the disclosed embodiments. Accordingly, one of skill in the art will realize that the inventive concept is intended to encompass all modifications and alternative arrangements within the spirit and scope of the inventive concept as set forth in the appended claims.

The presently claimed invention relates to the field of medical analytics. By processing information related to opioid prescription and providing analytics on the risk of opioid prescription, the invention improves the technological field of medicine by providing improved patient outcomes in pain management.

What is claimed is:

1. A method for identifying risks for opioid prescriptions, said method implemented by a processor included within an opioid analytics server, said opioid analytics server comprising said processor and a memory, said opioid analytics server in communication with at least a first electronic health records system, said method comprising:
receiving a risk assessment profile for analyzing opioid risks for a patient;
extracting a first set of health records from said first electronic health records system based on said risk assessment profile;
processing said first set of health records to determine a set of goals, a set of outcomes, and a set of risk factors;
determining, based on said set of goals, said set of outcomes, and said set of risk factors, a plurality of recommended tasks; and
providing a user interface comprising a first region containing information associated with said set of goals, said set of outcomes, a second region containing information associated with said set of risk factors, a third region associated with said plurality of tasks, and a fourth region configured to display a treatment tracking tool.

2. The method of claim 1, further comprising:
identifying a first opioid risk scoring tool, wherein said first opioid risk scoring tool comprises an algorithm used to analyze a level of risk associated with opioid risks for a patient;
applying said first opioid risk scoring tool to said first set of risk factors to determine an opioid risk score; and
providing an opioid risk score in said second region.

3. The method of claim 1, further comprising:
extracting a second set of health records from a secondary electronic health records system; and
processing said first set of health records with said second set of health records to determine said set of goals, said set of outcomes, and said set of risk factors.

4. The method of claim 1, further comprising:
identifying a prescription drug monitoring program associated with said risk assessment profile and said first set of health records; and
providing access to information from said prescription drug monitoring program from at least one of said first region and said second region.

5. The method of claim 1, further comprising:
determining a condition status associated with each of said set of goals and each of said set of outcomes; and
providing said user interface comprising said first region containing information associated with said set of goals and said set of outcomes, wherein said first region is provided including said condition status associated with each of said set of goals and each of said set of outcomes.

6. The method of claim 1, further comprising:
determining a condition status associated with each of said set of risk factors; and
providing said a user interface comprising said second region containing information associated with said set of risk factors, wherein said second region is provided including said condition status associated with each of said set of risk factors.

7. The method of claim 1, further comprising:
identifying at least one additional reporting tool; and
providing access to said at least one additional reporting tool using said user interface.

8. The method of claim 7, further comprising:
applying said first set of health records to said at least one reporting tool to generate a trend report; and
providing access to said trend report using said user interface.

9. The method of claim 8, further comprising:
generating said trend report, wherein said trend report includes a first report region indicating patient prescriptions over a time period, a second report region indicating patient critical events over said time period, and a third report region indicating information associated with said set of goals and said set of outcomes over said time period.

10. The method of claim 1, wherein providing the fourth region configured to display a treatment tracking tool further comprises:
extracting a second set of health records from a first electronic health records system based on a risk assessment profile;
determining from the second set of health records, a first listing of past treatment approaches for chronic pain, and a second listing of current treatment approaches for chronic pain;
analyzing the first listing and the second listing to determine a third listing of future treatment approaches; and
providing the treatment tracking tool within the fourth region, wherein the treatment tracking tool is configured to present at least the first, second, and third listing and a fourth listing of identified risks.

11. A system for identifying risks of opioid prescriptions comprising:
an opioid analytics server having a processor and a memory; and
a first electronic health records system in communication with said opioid analytics server, wherein said processor is configured to:
receive a risk assessment profile for analyzing opioid risks for a patient;
extract a first set of health records from said first electronic health records system based on said risk assessment profile;
process said first set of health records to determine a set of goals, a set of outcomes, and a set of risk factors;
determine, based on said set of goals, said set of outcomes, and said set of risk factors, a plurality of recommended tasks; and
provide a user interface comprising a first region containing information associated with said set of goals and said set of outcomes, a second region containing information associated with said set of risk factors, a third region associated with said plurality of tasks, and a fourth region configured to display a treatment tracking tool.

12. The system of claim 11, wherein said processor is configured to:
identify a first opioid risk scoring tool, wherein said first opioid risk scoring tool comprises an algorithm used to analyze a level of risk associated with opioid risks for a patient;
apply said first opioid risk scoring tool to said first set of risk factors to determine an opioid risk score; and
provide an opioid risk score in said second region.

13. The system of claim 11, wherein said processor is configured to:
extract a second set of health records from a secondary electronic health records system; and
process said first set of health records with said second set of health records to determine said set of goals, said set of outcomes, and said set of risk factors.

14. The system of claim 11, wherein said processor is configured to:
identify a prescription drug monitoring program associated with said risk assessment profile and said first set of health records; and
provide access to information from said prescription drug monitoring program from at least one of said first region and said second region.

15. The system of claim 11, wherein said processor is configured to:
determine a condition status associated with each of said set of goals and each of said set of outcomes; and
provide said user interface comprising said first region containing information associated with said set of goals and said set of outcomes, wherein said first region is provided including said condition status associated with each of said set of goals and each of said set of outcomes.

16. The system of claim 11, wherein said processor is configured to:
determine a condition status associated with each of said set of risk factors; and
provide said a user interface comprising said second region containing information associated with said set of risk factors, wherein said second region is provided including said condition status associated with each of said set of risk factors.

17. The system of claim 11, wherein said processor is configured to:
identify at least one additional reporting tool; and
provide access to said at least one additional reporting tool using said user interface.

18. The system of claim 17, wherein said processor is configured to:
apply said first set of health records to said at least one reporting tool to generate a trend report; and
provide access to said trend report using said user interface.

19. The system of claim 18, wherein said processor is configured to:
generate said trend report, wherein said trend report includes a first report region indicating patient prescriptions over a time period, a second report region indicating patient critical events over said time period, and a third report region indicating information associated with said set of goals and said set of outcomes over said time period.

20. The system of claim 11, wherein said processor is configured to:
extract a second set of health records from a first electronic health records system based on a risk assessment profile;
determine from the second set of health records, a first listing of past treatment approaches for chronic pain, and a second listing of current treatment approaches for chronic pain;
analyze the first listing and the second listing to determine a third listing of future treatment approaches; and
provide the treatment tracking tool within the fourth region, wherein the treatment tracking tool is configured to present at least the first, second, and third listing and a fourth listing of identified risks.

21. An opioid analytics server for identifying risks for opioid prescriptions, said opioid analytics server comprising a processor and a memory, said opioid analytics server in communication with at least a first electronic health records system, said processor is configured to:
receive a risk assessment profile for analyzing opioid risks for a patient;
extract a first set of health records from said first electronic health records system based on said risk assessment profile;
process said first set of health records to determine a set of goals, a set of outcomes and a set of risk factors;
determine, based on said set of goals, said set of outcomes, and said set of risk factors, a plurality of recommended tasks; and
provide a user interface comprising a first region containing information associated with said set of goals and said set of outcomes, a second region containing information associated with said set of risk factors, a third region associated with said plurality of tasks, and a fourth region configured to display a treatment tracking tool.

22. The opioid analytics server of claim 21, wherein said processor is configured to:
identify a first opioid risk scoring tool, wherein said first opioid risk scoring tool comprises an algorithm used to analyze a level of risk associated with opioid risks for a patient;
apply said first opioid risk scoring tool to said first set of risk factors to determine an opioid risk score; and
provide an opioid risk score in said second region.

23. The opioid analytics server of claim 21, wherein said processor is configured to:
extract a second set of health records from a secondary electronic health records system; and
process said first set of health records with said second set of health records to determine said set of goals, said set of outcomes, and said set of risk factors.

24. The opioid analytics server of claim 21, wherein said processor is configured to:
identify a prescription drug monitoring program associated with said risk assessment profile and said first set of health records; and
provide access to information from said prescription drug monitoring program from at least one of said first region and said second region.

25. The opioid analytics server of claim 21, wherein said processor is configured to:
determine a condition status associated with each of said set of goals and each of said set of outcomes; and
provide said user interface comprising said first region containing information associated with said set of goals and said set of outcomes, wherein said first region is provided including said condition status associated with each of said set of goals and each of said set of outcomes.

26. The opioid analytics server of claim 21, wherein said processor is configured to:
determine a condition status associated with each of said set of risk factors; and
provide said a user interface comprising said second region containing information associated with said set of risk factors, wherein said second region is provided including said condition status associated with each of said set of risk factors.

27. The opioid analytics server of claim 21, wherein said processor is configured to:
identify at least one additional reporting tool; and
provide access to said at least one additional reporting tool using said user interface.

28. The opioid analytics server of claim 27, wherein said processor is configured to:
apply said first set of health records to said at least one reporting tool to generate a trend report; and
provide access to said trend report using said user interface.

29. The opioid analytics server of claim 28, wherein said processor is configured to:
generate said trend report, wherein said trend report includes a first report region indicating patient prescriptions over a time period, a second report region indicating patient critical events over said time period, and a third report region indicating information associated with said set of goals and said set of outcomes over said time period.

30. The opioid analytics server of claim 21, wherein said processor is configured to:
extract a second set of health records from a first electronic health records system based on a risk assessment profile;
determine from the second set of health records, a first listing of past treatment approaches for chronic pain, and a second listing of current treatment approaches for chronic pain;
analyze the first listing and the second listing to determine a third listing of future treatment approaches; and
provide the treatment tracking tool within the fourth region, wherein the treatment tracking tool is configured to present at least the first, second, and third listing and a fourth listing of identified risks.
